(19) United States
(12) Patent Application Publication
(10) Pub. No.: US 2003/0203388 A1
(43) Pub. Date: Oct. 30, 2003

(54) Method for determining genotoxicity

(75) Inventor: Jiri Aubrecht, Stonington, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/422,377

(22) Filed: Apr. 24, 2003

Related U.S. Application Data
(60) Provisional application No. 60/375,784, filed on Apr. 26, 2002.

(51) Int. Cl. C12Q 1/68; C12N 15/85

(52) U.S. Cl. 435/6; 435/455

(57) ABSTRACT

The invention relates to methods for characterizing the genotoxicity of an agent.

### CROSS REFERENCE TO RELATED APPLICATION

This application claims the benefit of U.S. Provisional Application No. 60/375,784 filed Apr. 26, 2002.

### FIELD OF THE INVENTION

The present invention relates to methods for characterizing the genotoxicity of an agent.

### BACKGROUND OF THE INVENTION

Genetic toxicology testing in the pharmaceutical industry provides an assessment of genotoxic risk associated with the use of drugs. Since the beginning of genotoxicity testing in the early 1970s, many different test systems have been developed and used. No single conventional test is capable of detecting all genotoxic agents. Therefore, genotoxic evaluation of pharmaceutical compounds involves the use of a standard battery of in vitro and in vivo assays. See ICH (1997) Harmonized tripartite guideline, genotoxicity: a standard battery for genotoxicity testing of pharmaceuticals. Recommendations for adoption at step 4 of the ICH process on July 1997 by the ICH Steering Committee (final draft). These tests include bacterial reverse-mutation tests, in vitro tests for chromosomal damage (e.g., cytogenetic assays and in vitro mouse lymphoma thymidine kinase assay) and in vivo tests for chromosomal damage (e.g., rodent micronucleus test)

Molecular biology and recombinant technology provide additional methods by which genotoxicity may be measured. For example, differential gene expression technology may be used to study changes in gene expression of cells exposed to drug and chemical substances. Differential gene expression may be measured using various techniques known to those with skill in the art, including, gel electrophoresis and polynucleotide microarrays.

Genotoxic stress triggers a variety of biological responses including the transcriptional activation of genes regulating DNA repair, cell survival and cell death. Factors that may cause genotoxic stress include exposure to chemical and biological agents and radiation. GADD45 has been identified as a gene that is induced by DNA damaging factors such as ultraviolet and gamma radiation, the alkylating agent, methyl methanesulfonate (MMS), N-acetoxy-2-acetylaminofluorine and hydrogen peroxide. Fornace, A. J., et al. (1988) Proc. Natl. Acad. Sci. USA 85, 8800-8804; Papathanasiou, M. A., et al. (1991) C. Mol. Cell. Biol. 11, 1009-1016; and Takekawa, M. and Saito H. (1998) Cell 95(4), 521-530.

It has been reported that GADD45 may be induced by interleukin receptor ligands. Nakayama, K., et al. (1999) J. Biol. Chem. 274(35), 24766-24772; Brysk M. M., et al. (1995) J. Interferon Cytokine Res. 15(12), 1029-1035. The activation of GADD45 gene expression as a consequence of oxidative stress and of ischemia have also recently also been reported. See, Petrault, I., et al. (2002) Biochim Biophys Acta 1586(1), 92-98; Tchounwou, P. B., et al. (2001) Mol. Cell. Biochem. 222(1-2), 21-28; and Schmidt-Kastner, R., et al. (1998) Brain Res. Mol. Brain Res. 63(1), 79-97.

Cisplatin is a chemotherapeutic agent used in the treatment of cancer. Cisplatin damages DNA by forming DNA-protein cross-links. See, Costa, M., et al. (1997) J. Toxicol. Environ. Health 50(5), 433-449; and Zhitkovich, A. and Costa, M. (1992) Carcinogenesis 13(8),1485-1489.

Aubrecht, J., et al. (1999) Toxicol. Appl. Pharmacol. 154(3), 228-235 discloses that metallocene molecules containing vanadium, although structurally similar to cisplatin and causing cytotoxicity via apoptosis as does cisplatin, do not cause DNA double strand breaks measured by the yeast DEL recombination assay and do not activate GADD45 promoter activity.

PCT patent application publication WO 97/13877 and related U.S. Pat. No. 6,228,589 disclose methods for assessing the toxicity of a compound in a test organism by measuring gene expression profiles of selected tissues.

U.S. Pat. No. 5,811,231 describes methods and diagnostic kits for identifying and characterizing toxic compounds, wherein the methods and kits measure transcription or translation levels from genes linked to native eukaryotic stress promoters.

Rockett, John C., et al. (2000) Xenobiotica 30, 155-177 describes DNA arrays and methods of preparing and using DNA arrays and various applications for which DNA arrays may be used, including toxicological applications.

There exists a need to identify, characterize and understand the mechanism of action of toxicologically relevant genes in order to simplify the development, screening, and testing of new drug and chemical substances.

### SUMMARY OF THE INVENTION

One aspect of this invention provides methods of characterizing the DNA-interacting genotoxicity of an agent, comprising,

treating a mammal cell or a mammal with an agent; and

characterizing the DNA-interacting genotoxicity of said agent by determining the effect of said agent on expression in said mammal cell or mammal of at least one gene selected from the gene-set of Table 1.

In a preferred embodiment of the invention, said characterizing of genotoxicity comprises characterizing said agent as either:

a) DNA-interacting genotoxic, preferably DNA-cross-linking genotoxic, if the agent causes an increase in expression of at least one gene selected from said gene-set, or

b) of unknown DNA-interacting genotoxicity, preferably of unknown DNA-cross-linking genotoxicity, if the agent does not cause an increase in expression of at least one gene selected from said gene-set.

In a further preferred embodiment, said characterizing step comprises determining the effect of said agent on expression of at least two, preferably, at least three genes, selected from said gene-set. In a more preferred embodiment, said gene-set consists of gene accession numbers AV1 38783, AI847051, AF055638 and AI461837. In an even more preferred embodiment, said gene-set consists of AV138783, AI847051 and AF55638.

In an additional preferred embodiment, the effect of said agent on expression is determined for at least two genes comprising gene accession numbers AV1 38783 and AI847051, and even more preferably, on at least three genes comprising gene accession numbers AV138783, AI847051 and AF55638.

Another aspect of this invention provides methods of characterizing the genotoxicity of an agent, comprising:

treating a mammal cell or a mammal with an agent; and

determining the effect of said agent on expression in said mammal cell or mammal of at least one gene selected from:

gene accession numbers AV1 38783, AI847051, AF055638 and AI461837.

In a preferred embodiment of the methods of this invention, said mammal cell or mammal is a mouse cell or mouse, respectively.

The term “DNA-interacting genotoxicity” refers to the genotoxicity of an agent resulting from the direct physical interaction of the agent with the DNA of a cell. Such direct physical interaction includes, for example, direct chemical and physical interactions and reactions with DNA molecules, intercalations with DNA molecules, the formation of complexes with the DNA molecules, the formation of intra-DNA molecule and inter-DNA molecule cross-linking and the formation of DNA-protein cross-linking.

The term “DNA-cross-linking genotoxicity” refers to the genotoxicity of an agent resulting from intra-DNA molecule and inter-DNA molecule cross-linking or the formation of DNA-protein cross-linking, wherein said cross-linking is preferably through the formation of covalent bonding.

The term “gene-set” means the genes listed in Table 1 and their respective homologues and orthologues. The term “homolog” as used herein means a gene having at least 95 percent identity to the applicable gene in Table 1. The term “ortholog” as used herein means a gene of a species other than the species from the which applicable gene in Table 1 is derived, having at least 65 percent identity, preferably 75 percent identity, and even more preferably 85 percent identity, to the applicable gene in Table 1.

The term “genotoxicity” means being characterized as causing damage to the genetic material of a live cell. The damage to the DNA or RNA of a cell may include, for example, damage resulting from nucleotide or polynucleotide deletion, addition, point mutation, dimerization or recombination, DNA-DNA, RNA-RNA and RNA-DNA cross-linking, DNA-protein or RNA-protein cross-linking and DNA or RNA breakage or degradation. The damage may also include that which is evidenced by chromosomal numerical abnormalities, such as polyploidy or aneuploidy.

The term “increase in expression” when used in reference to the expression of one or more genes, means expression that represents at least a statistically significant increase as measured against a control.

“Nucleotide identity” as used herein refers to the sequence alignment of a nucleotide sequence calculated against another nucleotide sequence. Specifically, the term refers to the percentage of residue matches between at least two nucleotide sequences aligned using a standardized algorithm. Such an algorithm may insert gaps in the sequences being compared in a standardized and reproducible manner in order to optimize alignment between the sequences, thereby achieving a more meaningful comparison. Nucleotide identity between nucleotide sequences is preferably determined using the default parameters of the CLUSTAL W algorithm as incorporated into the version 5 of the MEGALIGN sequence alignment program. This program is part of the LASERGENE suite of molecular biological analysis programs (DNASTAR, Madison Wis.). CLUSTAL W is described in Thompson, J. D., et al. (1994) Nucleic Acids Research 22, 4673-4680.

“Nucleotide sequence” and “polynucleotide” refer to both DNA or RNA of genomic or synthetic origin which may be single-stranded or double-stranded and may represent a sense or an antisense strand. The term “complimentary nucleotide sequence” refers to a nucleotide sequence that anneals (binds) to a another nucleotide sequence according to the pairing of a guanidine nucleotide (G) with a cytidine nucleotide (C) and adenosine nucleotide (A) with thymidine nucleotide (T), except in RNA where a T is replaced with a uridine nucleotide (U) so that U binds with A.

### BRIEF DESCRIPTION OF THE DRAWINGS

FIG. 1 shows the results from an in vitro micronucleus assay of L5178Y cells treated with cisplatin and the effect of cisplatin treatment on viability of cells.

FIG. 2 shows the results from an in vitro micronucleus assay of L5178Y cells treated with sodium chloride and the effect of sodium chloride treatment on viability of cells.

FIG. 3 shows the effect on the expression of various genes as a result of cisplatin treatment. Results are expressed as a fold increase compared to a negative control.

### DETAILED DESCRIPTION OF THE INVENTION

It has been reported that GADD45 may be induced by factors such as ultraviolet and gamma radiation, the alkylating agent, methyl methanesulfonate (MMS), N-acetoxy-2-acetylaminofluorine and hydrogen peroxide as well as interleukine receptor ligands, oxidative stress and ischemia (See, Fornace, A. J., et al. (1988) Proc. Natl. Acad. Sci. USA 85, 8800-8804; Papathanasiou, M. A., et al. (1991) C. Mol. Cell. Biol. 11, 1009-1016; Takekawa, M. and Saito H. (1998) Cell 95(4), 521-530; Nakayama, K., et al. (1999) J. Biol. Chem. 274(35), 24766-24772; Brysk M. M., et al. (1995) J. Interferon Cytokine Res. 15(12), 1029-1035, Petrault, I., et al. (2002) Biochim Biophys Acta 1586(1), 92-98; Tchounwou, P. B., et al. (2001) Mol. Cell. Biochem. 222(1-2), 21-28; and Schmidt-Kastner, R., et al. (1998) Brain Res. Mol. Brain Res. 63(1), 79-97.

The results from the aforementioned publications infer that GADD45 is a general indicator of stress. This invention is based, in part, on the discovery that, although cisplatin and high concentrations of sodium chloride both show evidence of genotoxic stress, as demonstrated using a micronucleus assay, only cisplatin results in increased expression of certain genes known to be associated with genotoxic stress, GADD45β (gene accession number X54149) and GADD45 (gene accession number U00937). Such results suggest that GADD45β and GADD45 are indicative of a mechanism of DNA damage that involves direction interaction with DNA.

This invention is also based, in part, on the discovery that the expression of the genes having accession numbers AV138783, AI847051, AF055638 and AI461837 increase as a result of genotoxic stress.

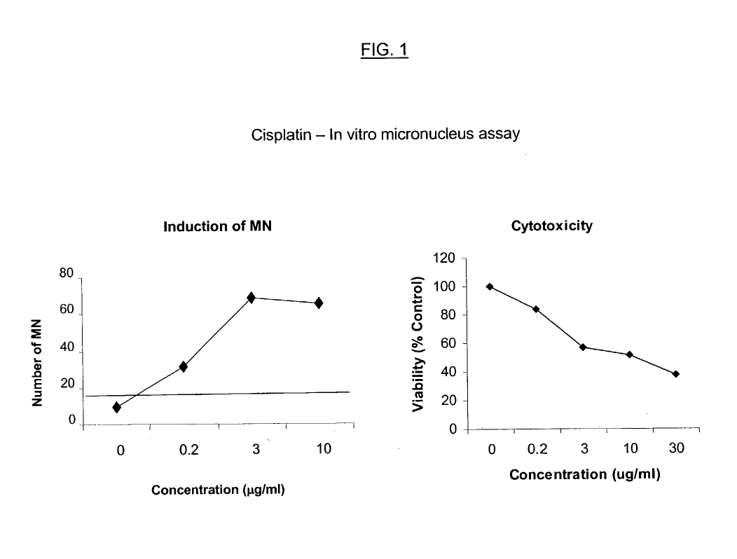

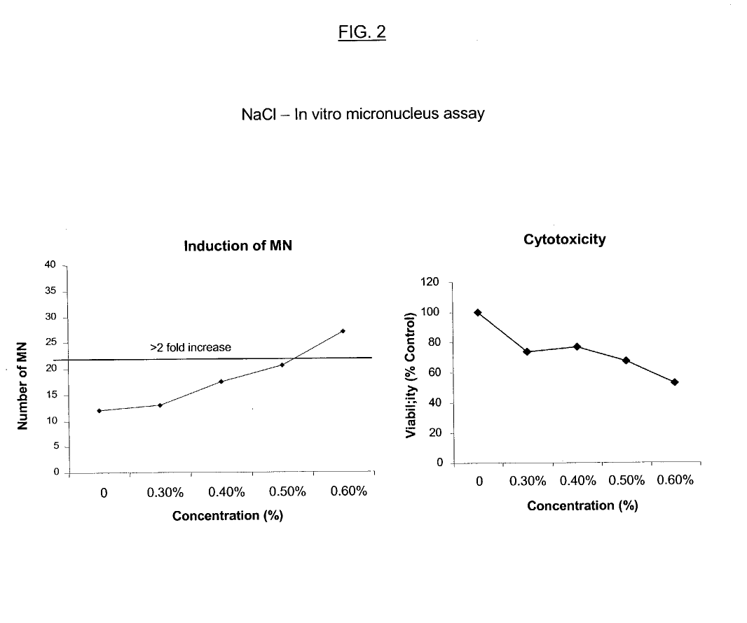

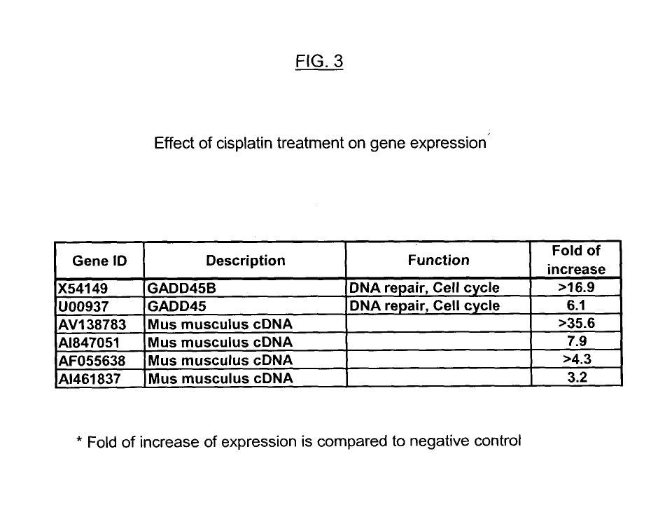

The effects of cisplatin and sodium chloride treatment of cells on micronuclei formation are illustrated in FIG. 1 and FIG. 2, respectively. The effect of cisplatin treatment on expression of the aforementioned genes is illustrated in FIG. 3. Sodium chloride had no effect on the level of expression of these genes.

Accordingly, one embodiment of the present invention provides methods using the genes listed in Table 1 for identifying agents that are genotoxic as a result of direct interaction with DNA. In another embodiment, the genes having accession numbers AV138783, AI847051, AF055638 and AI461837 are used to identify agents that are genotoxic.

It will be appreciated by those with skill in the art based upon the present disclosure, that any mammalian cell may be used in the practice of this invention. Mammalian cell lines that propagate indefinitely are preferred. The cells may be derived from any of a number of organs, including thymus, spleen, bone marrow, lymphocytes, liver, kidney, heart, testis, ovary, heart and skeletal muscle. Preferably, the cells are derived from liver, kidney or thymus. Such cells may be derived from any mammal, but preferably are derived from mouse, rat, dog or human, more preferably rat, mouse or human. As described below, it is preferable to match the species from which the cells are derived to the species of the gene fragments used as probes or microarray oligomers in detecting expression of the genes herein described.

A preferred cell line for use in the practice of this invention is L5178Y (contributed by Mobil Oil Corporation, ATCC No. CRL 9518, American Type Tissue Culture collection (ATCC), Manassas, Va.). Other suitable cells lines are available, for example, from the ATCC and other depositories well known to those with skill in the art.

In a preferred embodiment utilizing a mammalian cell line, the cells are prepared prior to treatment with a test agent by developing a culture of the cells. As is well known by those with skill in the art, culture conditions will vary depending upon the cell type used. For example, when using L5178Y cells, the cells are routinely grown until they reach a density of less than about 7×10⁵-1×10⁶ cells/ml. Prior to treatment with the test agent, the cells are diluted to about 5×10⁵/ml and treated with varying concentrations of the test agent. As those with skill in the art will appreciate based upon the present disclosure, for any given test agent the concentration range will vary depending upon the cell type and test agent used, but should be between zero and that concentration which results in cell death. The period of exposure will generally range from between about five minutes to about 48 hours, preferably about two hours to about 32 hours.

Some agents that are not toxic to mammals in their native form, may become genotoxic after being processed by the liver. Thus, according to one embodiment of this invention, an agent to be tested is first pre-treated with a liver extract, such as an S9 liver extract as generally described by Vennitt, S., et al. (1984) Mutagenicity Testing—A Practical Approach, IRL Press, Oxford, England, 52-57. Alternatively, cells utilized in the practice of the invention may be co-cultured, for example, with hepatocytes or cells derived from a hepatocyte cell line. However, if the cells utilized for the methods of the invention are themselves hepatocytes or cells derived from a hepatocyte cell line, then pre-treatment or co-culturing is not generally necessary.

Any mammal may be used in the practice of this invention. Preferably, such mammal is a rodent, more preferably a rat or mouse, even more preferably, a mouse. As described below, it is preferable to match the species of mammal to the species of the gene fragments used as probes or microarray oligomers in detecting expression of the genes herein described.

It will be appreciated by those with skill in the art based upon the present disclosure that, in the practice of the invention, the determination of gene expression of the genes described herein in a mammal is performed, following treatment of the mammal with a test agent, on the cells or tissue of one or more organs of the mammal. The cells or tissue may be derived from any of a number of organs of the mammal, including thymus, spleen, bone marrow, white blood cells, liver, kidney, heart, testis, heart and skeletal muscle. Preferably, the cells or tissue are derived from liver, kidney or bone marrow.

In the method aspects of this invention, the effect of a test agent on expression of toxicologically relevant genes is analyzed. The detection of changes in gene expression is preferably performed by measuring messenger RNA (mRNA) expression of a gene by methods well known to those with skill in the art based upon the present disclosure. For example, one method that may be employed to measure mRNA expression involves polymerase chain reaction (PCR) and gel electrophoresis to detect differentially expressed genes. For example, the product from PCR synthesis may be subjected to gel electrophoresis, and bands produced by two or more mRNA populations may be compared. Bands present on an autoradiograph of one gel from one mRNA population, and not present on another, correspond to the presence of a particular mRNA in one population and not in the other, and thus indicate a gene that is likely to be differentially expressed. (See, Williams, J. G. (1990) NucL Acids Res. 18, 6531; Welsh, J., et al., (1990) NucL Acids Res., 18, 7213; Woodward, S. R., (1992) Mamm. Genome, 3, 73; and Nadeau, J. H. (1992) Mamm. Genome 3, 55; Liang, P. et al., (1992) Science 257, 967; Welsh, J. et al. (1992) Nucl. Acid Res. 20, 4965; Liang, P., et al. (1993) NucL Acids Res. 3269, 1993; and U.S. Pat. Nos. 6,114,114 and 6,228,589).

As used herein, the terms “arrays” and “microarrays” refer to an array of distinct polynucleotides or oligonucleotides synthesized on a substrate, such as paper, nylon or other type of membrane, filter, chip, glass slide, or any other suitable solid support. In a preferred embodiment, microarrays may be prepared and used according to the methods described in U.S. Pat. No. 5,837,832, PCT application WO95/11995, Lockhart et al. (1996) Nat. Biotech. 14:1675-1680 and Schena, M. et al. (1996) Proc. Natl. Acad. Sci._{—}93:10614-10619, all of which are incorporated herein in their entirety by reference. In other embodiments, such arrays are produced by the methods described by Brown et al., U.S. Pat. No. 5,807,522.

A preferred method for detecting mRNA expression is by using microarrays. The process of isolating mRNA from cells or tissues exposed to a stimulus (e.g., drugs or chemicals) and analyzing the expression with gel electrophoresis can be laborious and tedious. To that end, microarray technology provides a faster and-more efficient method of detecting differential gene expression. Differential gene expression analysis by microarrays involves nucleotides immobilized on a substrate whereby nucleotides from cells that have been exposed to a stimulus can be contacted with the immobilized nucleotides to generate a hybridization pattern. See, for example, Diehn M, et al. (1993) Nat Genet. 25(1), 58-62; Scherf, U., et al. Nat Genet. 24(3): 236-44 (1993); Hayward R. E., et al. (1993) Mol. Microbiol. 35(1), 6-14; Johannes G., et al. (1993) Proc. Natl. Acad. Sci. USA 96(23), 13118-23.

A microarray is preferably composed of a large number of unique, single-stranded nucleic acid sequences, usually either synthetic antisense oligonucleotides or fragments of cDNAs, fixed to a solid support. The oligonucleotides are preferably about 6-60 nucleotides in length, more preferably 15-30 nucleotides in length, and most preferably about 20-25 nucleotides in length. For a certain type of microarray or detection kit, it may be preferable to use oligonucleotides that are only 7-20 nucleotides in length. The microarray or detection kit may contain oligonucleotides that cover the known 5′, or 3′, sequence, sequential oligonucleotides which cover the full length sequence; or unique oligonucleotides selected from particular areas along the length of the sequence. Polynucleotides used in the microarray or detection kit may be oligonucleotides that are specific to a gene or genes of interest.

To produce oligonucleotides to a known sequence for a microarray or detection kit, the genes of interest are typically examined using a computer algorithm which starts at the 5′ or at the 3′ end of the nucleotide sequence. Typical algorithms may then be used to identify oligomers of defined length that are unique to the gene, have a GC content within a range suitable for hybridization, and lack predicted secondary structure that may interfere with hybridization. In certain situations it may be appropriate to use pairs of oligonucleotides on a microarray or detection kit. The “pairs” are identical, with the exception of one nucleotide, preferably located in the center of the sequence. The second oligonucleotide in the pair (mismatched by one) serves as a control. The number of oligonucleotide pairs may range from two to one million. The oligomers are synthesized at designated areas on a substrate using a light-directed chemical process. The substrate may be paper, nylon or other type of membrane, filter, chip, glass slide or any other suitable solid support.

An oligonucleotide may be synthesized on the surface of the substrate by using a chemical coupling procedure and an ink jet application apparatus, as described in PCT application WO95/251116. A “gridded” array analogous to a dot (or slot) blot may be used to arrange and link cDNA fragments or oligonucleotides to the surface of a substrate using a vacuum system, thermal, LTV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more oligonucleotides, or any other number between two and one million which lends itself to the efficient use of commercially available instrumentation.

As will be appreciated by those with skill in the art, it is preferable to match the species from which the expression samples are derived to the species of the gene fragments oligomers used in the preparation of microarrays. For example, if expression samples are prepared from a human cell line, then a microarray containing human gene fragments is preferably used containing the applicable gene from the gene-set in Table 1 or its applicable ortholog.

To conduct sample analysis using a microarray, hybridization probes are prepared from the RNA of the biological sample to be tested. For example, the mRNA may be isolated, and cDNA produced and used as a template to make antisense RNA (aRNA). The aRNA may then be labeled by amplifying in the presence of labeled nucleotides (e.g., fluorescently labeled nucleotides). Labeled probes are incubated with the microarray so that the probe sequences hybridize to complementary oligonucleotides of the microarray. Incubation conditions are adjusted so that hybridization occurs with precise complementary matches or with various degrees of less complementary matching.

After removal of non-hybridized probes, a scanner may be used to determine the levels and patterns of fluorescence. The scanned image may then be examined to determine degree of complementarity and the relative abundance of each oligonucleotide sequence on the microarray.

In a preferred embodiment of the invention, microarrays are used to detect the expression of the genes listed in Table 1. The method comprises incubating a test sample with one or more nucleic acid molecules and assaying for binding of the nucleic acid molecule with components within the test sample. Such assays will typically involve arrays comprising many genes, at least one of which is a gene listed in Table 1 of the present invention.

Conditions for incubating a nucleic acid molecule with a test sample vary. Incubation conditions depend on the format employed in the assay, the detection methods employed and the type and nature of the nucleic acid molecule used in the assay. One skilled in the art will recognize that any one of the commonly available hybridization, amplification or array assay formats can readily be adapted to employ fragments of the genes listed in Table 1. Examples of such assays can be found in Chard, T (1986), An Introduction to Radiaimmunoassay and Related Techniques, Elsevier Science Publishers, Amsterdam, The Netherlands; Bullock, G. R. et al. Techniques in Immunocytochemistry, Academic Press, Orlando, Fla. Vol. 1 (1982), Vol-2 (1983), Vol-3 (1985); Tijssen, P. (1985) Practice and Theory of Enzyme Immunoassays: Laboratory Techniques in Biochemistry and Molecular Biology, Elsevier Science Publishers, Amsterdam, The Netherlands.

Methods for preparing nucleic acid extracts of cells in the practice of the present invention are well known in art and can be readily be adapted in order to obtain a sample that is compatible with the system utilized.

TABLE 1

| Accession Number | Name Designation |
|---|---|
| X54149 | GADD45μ |
| U00937 | GADD45 |
| AV138783 | |
| A1847051 | |
| AF055638 | |
| A1461837 | |

The disclosures of all patents, applications, publications and documents, for example brochures or technical bulletins, cited herein, are hereby expressly incorporated by reference in their entirety.

It is believed that one skilled in the art can, using the present description, including the examples, drawings, sequence listings and attendant claims, utilize the present invention to its fullest extent. The following Examples are to be construed as merely illustrative of the practice of the invention and not limitative of the remainder of the disclosure in any manner whatsoever.

### EXAMPLES

### Example 1

### Treatment of Cells with Test Substances

Mouse lymphoma cell line L5178Y (ATCC No. CRL 9518, ATCC) were cultivated in RPMI medium (Invitrogen Life Technologies, Inc., Carlsbad, Calif.) supplemented with 10% fetal bovine serum and antibiotics penicillin and streptomycin at 37° C. Cells from exponentially growing cultures were seeded at a concentration 3×10⁵ cells/ml and incubated for 24 hours and maintained below 1×10⁶ cells/ml. The cells were then diluted to 5×10⁵ cells/ml and treated for four hours using either cisplatin in RPMI medium, at concentrations of 0, 0.2, 3, 10 and 30 μg/ml, or RPMI medium containing elevated RPMI concentrations of 0, 0.30, 0.40, 0.50 and 0.60 percent above the sodium chloride concentration of RPMI (0.9%). After the treatment, the cells were washed in PBS (Invitrogen Life Technologies, Inc., Carlsbad, Calif.). Half of the cells where then immediately frozen and stored at −80° C. prior to gene expression analysis, as described in Example 2 below. The other half was allowed to recover in a fresh growth medium for 20 hours. Cell viability was determined based on a cell count. Cell viability results appear in FIG. 1 and FIG. 2 as a ratio between treated versus vehicle control. The cells were then harvested for use in the in vitro micronucleus assay of Example 3 below.

Example 1 illustrates the treatment of cells with a test substance in the practice of the methods of the present invention.

### Example 2

### Gene Expression Analysis

Gene expression was measured using Affymetrix GeneChip® technology (Affymetrix, Inc., Santa Clara, Calif.) using standard protocols available from Affymetrix (see, GeneChip® Expression Analysis, Technical Manual, Rev. 2 (2001), Affymetrix, Inc., available at http://www.affymetrix.com/pdf/expression_manual.pdf). After treatment of L5178Y cells according to Example 1, total RNA was isolated for the cells using a RNEasy™ kit (Qiagen, Valencia, Calif.). Hybridization probe was prepared using total RNA and following standard procedures recommended by Affymetrix (see, GeneChip® Expression Analysis, Technical Manual). The probe was hybridized to Affymetrix mouse microarray chip (U74) using standard procedures recommended by Affymetrix. The hybridized chips were scanned using a Hewlett Packard GeneArray™ Scanner, (Hewlett Packard Company, Palo Alto, Calif.) and the resulting data were evaluated using Affymetrix Microarray suite software (Affymetrix). A minimum of three independent experiments for each experimental condition was performed.

Example 2 illustrates the use of microarrays for measuring gene expression of the cells treated according to Example 1 in the practice the methods of the present invention.

### Example 3

### Micronucleus Assay

Treated cells prepared according to Example 1 were attached to glass slides by dropping them onto the glass slides using a pipette after hypotonic treatment with 75 mM KCl (Sigma-Aldrich Corp., St Louis, Mo.) and fixation with 3:1 (v/v) MeOH:acetic acid (JT Baker, Inc., Phillipsburg, N.J.). The cells were then stained using 31.25 μg/ml solution of acridine orange (Molecular Probes, Inc., Eugene, Oreg.).

Micronuclei were counted using a Axiokop 2 fluorescent microscopy (Carl Zeiss Microlmaging, Inc., Thornwood, N.Y.). The results of the micronuleus assay are shown in FIGS. 1 and 2.

Example 3 illustrates the micronucleus assay used to confirm the methods of the present invention.

1. A method of characterizing an agent, comprising,
treating a mammal cell or a mammal with an agent; and
characterizing the DNA-interacting genotoxicity of said agent by determining the effect of said agent on expression in said mammal cell or mammal of at least one gene selected from the gene-set of Table 1.

2. A method of claim 1 wherein said characterizing of genotoxicity comprises characterizing said agent as either:
a) DNA-interacting genotoxic, if the agent causes an increase in expression of at least one gene selected from said gene-set, or
b) of unknown DNA-interacting genotoxicity, if the agent does not cause an increase in expression of at least one gene selected from said gene-set.

3. A method of claim 2 wherein said characterizing of genotoxicity comprises characterizing said agent as either:
a) DNA-cross-linking genotoxic, if the agent causes an increase in expression of at least one gene selected from said gene-set, or
b) of unknown DNA-cross-linking genotoxicity, if the agent does not cause an increase in expression of at least one gene selected from said gene-set.

4. A method of claim 1 wherein said characterizing step comprises determining the effect of said agent on expression of at least two genes selected from said gene-set.

5. A method of claim 2 wherein said characterizing step comprises determining the effect of said agent on expression of at least two genes selected from said gene-set.

6. A method of claim 1 wherein said characterizing step comprises determining the effect of said agent on expression of at least three gene selected from said gene-set.

7. A method of claim 2 wherein said characterizing step comprises determining the effect of said agent on expression of at least three gene selected from said gene-set.

8. A method of claim 1 wherein the effect of said agent on expression is determined for at least two genes comprising gene accession numbers AV138783 and AI847051.

9. A method of claim 2 wherein the effect of said agent on expression is determined for at least two genes comprising gene accession numbers AV138783 and AI847051.

10. A method of claim 1 wherein the effect of said agent on expression is determined for at least three genes comprising gene accession numbers AV138783, AI847051 and AF55638.

11. A method of claim 2 wherein the effect of said agent on expression is determined for at least three genes comprising gene accession numbers AV138783, AI847051 and AF55638.

12. A method of characterizing the genotoxicity of an agent, comprising:
treating a mammal cell or a mammal with an agent; and
determining the effect of said agent on expression in said mammal cell or mammal of at least one gene selected from gene accession numbers AV138783, AI847051, AF055638 and AI461837.

13. A method of claim 1 wherein said mammal cell or mammal is a mouse cell or a mouse.

14. A method of claim 2 wherein said mammal cell or mammal is a mouse cell or a mouse.

15. A method of claim 3 wherein said mammal cell or mammal is a mouse cell or a mouse.

16. A method of claim 12 wherein said mammal cell or mammal is a mouse cell or a mouse.
